(19) United States
(12) Patent Application Publication
(10) Pub. No.: US 2026/0066045 A1
(43) Pub. Date: Mar. 5, 2026

(54) SYSTEMS AND METHODS FOR RANKING ACTIVE AGENTS

(71) Applicant: L'OREAL, Paris (FR)

(72) Inventors: Patrick Morley WILLOUGHBY, New York, NY (US); I-Chien LIAO, Priceton, NJ (US); Rebecca BARRESI, Robbinsville, NJ (US); Ying CHEN, Montville, NJ (US)

(21) Appl. No.: 18/819,946

(22) Filed: Aug. 29, 2024

(51) Int. Cl. G16B 25/10; G01N 33/50

(52) U.S. Cl. CPC G16B 25/10; G01N 33/5023; G01N 2570/00

(57) ABSTRACT

Systems, apparatuses, methods, and computer program products are disclosed for ranking a plurality of active agents applied to cells of interest. In an embodiment, the method may include a multi-marker genetic or OMICs analysis of a plurality of portions of treated cells, each of the portions of treated cells being treated with one or more of a plurality of active agents. The method may include determining a rank for each of the plurality of active agents for one or more selected characteristics based on application of the multi-marker OMICS analysis to an expression model for the one or more selected characteristics. The method may include determining one or more adjusted formula of a mixture including one or more of the plurality of active agents based on the rank.

### TECHNOLOGICAL FIELD

Example embodiments of the present disclosure relate generally to systems and methods for ranking active agents when applied to cell samples and to systems and methods for determining a formula or active agent efficacy based on the rank.

### BACKGROUND

Currently, ribonucleic acid (RNA) analysis, as well as other OMICS analysis or sequencing, produces varying types of charts. These charts may include heatmaps and/or other types of charts representing different types of gene expressions, such as Z-score gene expression or a differential gene expression. Further, the gene set may be a senescence curated gene set, which may be biased.

Additionally, the output of OMICS analysis is, typically, not easily interpretable given transcriptomic heterogeneity and variability. In other words, interpreting the resulting output from OMICS analysis may require a user with a higher than typical level of expertise. Further, such an interpretation, even with an expert user, still takes significant time and effort, as well as potentially including bias. Thus, even if a user correctly interprets the resulting analysis, user bias may cause incorrect interpretations.

### BRIEF SUMMARY

As noted, typical ribonucleic acid (RNA) analysis and/or other OMICS analysis results in an output that is difficult to interpret and may include bias. Thus, there is felt a need to limit and solve the aforementioned problems and drawbacks and provide systems and methods for scoring and/or ranking active agents when applied to cell samples and to systems and methods for determining adjusted and/or suggested formula based on those scores and/or rank, thus increasing interpretability and enabling faster and meaningful adjustments to product formula. Such systems and methods, as well as apparatus and computer programs or products, may obtain data from OMICS sequencing of cells (for example using genomics sequencing, transcriptomics sequencing, proteomics sequencing, epigenomics sequencing, lipidomics sequencing, or metabolomics sequencing or other types of OMICS sequencing or other tests) each applied with one of a plurality of active agents at varying concentrations. In other embodiments, each cell sample may be applied with a mixture including one or more active agents. The OMICS sequencing may be based on one or more characteristics of the cells, one or more gene characteristics, senescence scores, single cell transcriptomic measurements using RNA Multiplexed error-robust fluorescence in situ hybridization (MERFISH), and/or on another cell characteristic test, as will be understood by one skilled in the art. Once the data has been obtained, such systems and methods may score and/or rank each active agent and/or concentration of each active agent as applied to the cells. Based on the scores and/or rank, the systems and methods may determine the efficacy of selected mixture or formula for a product including one or more of the ranked active agents, as well as suggest or provide an adjustment to the selected mixture or formula.

Accordingly, an embodiment of the disclosure is directed to a method for ranking and/or scoring a plurality of active agents applied to cells of interest. The method may include identifying cells of interest of a subject. The method may include iteratively treating different portions of cells with one of the plurality of active agents and/or, in other embodiments, a mixture including a plurality of the active agents. The method may include analyzing, via an OMICS sequencer, each portion of treated cells to produce a multi-marker OMICS analysis. The method may include determining a rank for each of the plurality of active agents for one or more selected characteristics based on application of the multi-marker OMICS analysis to a gene expression model for the one or more selected characteristics. The method may include determining one or more adjusted formula of a mixture including one or more of the plurality of active agents based on the rank.

In another embodiment, determining the ranking may include converting the multi-marker OMICS analysis to an array of values, removing identifiers from the array of values, determining an average of the array of values and a number of instances in the array of values, and applying the array of values to an assigned pair or unpaired ranked test to generate a rank for each value in the array of values based on a signature of the one or more selected characteristics, and re-assigning the identifiers to the array of values. In an embodiment, the multi-marker OMICS analysis may include one of transcriptomics, proteomics, epigenomics, genomics, lipidomics, or metabolomics.

In another embodiment, the plurality of active agents may include a plurality of cosmetic compounds. The cosmetic compounds may include one or more of peptides, molecules, extracts, chemicals, or devices. Further, the one or more of the plurality of active agents may include naturally occurring peptides. The devices may include one or more of a device that uses radiofrequency, fractional lasers, ablative lasers, non-ablative lasers, or micro needling. In an embodiment, the cells of interest may include one or more of skin cells, hair cells, scalp cells, tissue cells, reconstructed tissue, or ex vivo skin.

In another embodiment, the method may include, in response to determination of the ranking for each of the plurality of active agents and/or mixtures including a plurality of active agents, generating a user interface. The method may include ranking each of the plurality of active agents based on the ranking. The method may include displaying each of the plurality of agents based on ranking. The method may include displaying each of the one or more adjusted formula.

In another embodiment, the one or more selected characteristics may include one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health.

Another embodiment of the disclosure is directed to an apparatus for ranking a plurality of active agents applied to cells of interest. The apparatus may include an analysis circuitry. The analysis circuitry may be configured to obtain a multi-marker OMICS analysis of a plurality of portions of treated cells, each of the portions of treated cells being treated with one or more of a plurality of active agents. The apparatus may include a ranking circuitry. The ranking circuitry may be configured to determine a rank for each of the plurality of active agents for one or more selected characteristics based on application of the multi-marker OMICS analysis to a gene expression model for the one or more selected characteristics. The ranking circuitry may be configured to determine one or more adjusted formula of a mixture including one or more of the plurality of active agents based on the ranking.

In another embodiment, the apparatus may include an OMICS sequencer. In an embodiment, the OMICS sequencer may be a RNA sequencer or other types of sequencers. The OMICS sequencer may be configured to analyze cells to produce the multi-marker OMICS analysis. The analysis circuitry may communicatively connect to the OMICS sequencer and the analysis circuitry may be further configured to, in response to reception of cells of interest at the OMICS sequencer, prompt the OMICS sequencer to analyze the cells of interest.

In an embodiment, a user interface may be communicatively connected to the analysis circuitry and the analysis circuitry may obtain the multi-marker OMICS analysis from the user interface.

In another embodiment, the active agents may include naturally derived peptides, wherein the cells of interest comprise skin cells, and wherein the one or more selected characteristics comprise skin barrier health.

Another embodiment of the disclosure is directed to a computer program product for ranking a plurality of active agents applied to cells of interest. The computer program product may include a non-transitory machine-readable storage medium storing software instructions that, when executed, cause an apparatus to obtain a multi-marker OMICS analysis of a plurality of portions of treated cells, each of the portions of treated cells being treated with one or more of a plurality of active agents. Execution of the software instructions may further cause the apparatus to determine a rank for each of the plurality of active agents for one or more selected characteristics based on application of the multi-marker OMICS analysis to a gene expression model for the one or more selected characteristics.

In another embodiment, the non-transitory machine-readable storage medium may store additional software instructions that, when executed, cause an apparatus to determine one or more adjusted formula of a mixture including one or more of the plurality of active agents based on the ranking.

In another embodiment, the mixture may comprise a topical mixture applicable to skin and including one or more of the plurality of active agents and other materials. In another embodiment, a multi-marker OMICS analysis may include one or more of a heatmap, a cell by gene matrix, sample by gene matrix, cell by OMICS data matrix, sample by OMICS data matrix, or cell type distribution.

Another embodiment of the disclosure is directed to a system for ranking a plurality of active agents applied to cells of interest. The system may include an OMICS analyzer or sequencer. In response to reception of a plurality of cell samples each applied with a different one of a plurality of active agents, the OMICS analyzer or sequencer may analyze the each of the plurality of cell samples to produce a multi-marker OMICS analysis. The system may also include a computing device. The computing device may include a processor and memory. The memory may store instructions that, when executed, cause the processor to determine a ranking for each of the plurality of active agents for one or more selected characteristics based on application of the multi-marker OMICS analysis to a gene expression model for the one or more selected characteristics and then determine one or more adjusted formula of a mixture including one or more of the plurality of active agents based on the ranking.

The foregoing brief summary is provided merely for purposes of summarizing example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized above, some of which will be described in further detail below.

### BRIEF DESCRIPTION OF THE FIGURES

Having described certain example embodiments of the present disclosure in general terms above, reference will now be made to the accompanying drawings, which are not necessarily drawn to scale. Some embodiments may include fewer or more components than those shown in the figures.

FIG. 1A and FIG. 1B illustrate systems in which some example embodiments may be used for scoring and/or ranking each of a plurality of active agents applied to cells.

FIG. 2 illustrates a schematic block diagram of example circuitry embodying a device that may perform various operations in accordance with some example embodiments described herein.

FIG. 3A and FIG. 3B illustrate example user interfaces used in some example embodiments described herein.

FIG. 4 illustrates an example flowchart for scoring and/or ranking each of a plurality of active agents applied to cells, in accordance with some example embodiments described herein.

### DETAILED DESCRIPTION

Some embodiments of the present disclosure will now be described more fully hereinafter with reference to the accompanying figures, in which some, but not all, embodiments of the disclosures are shown. Indeed, these disclosures may be embodied in many different forms and should not be construed as limited to the embodiments set forth herein; rather, these embodiments are provided so that this disclosure will satisfy applicable legal requirements.

The term “computing device” is used herein to refer to any one or all of programmable logic controllers (PLCs), programmable automation controllers (PACs), industrial computers, desktop computers, personal data assistants (PDAs), laptop computers, tablet computers, smart books, palm-top computers, personal computers, smartphones, wearable devices (such as headsets, smartwatches, or the like), and similar electronic devices equipped with at least a processor and any other physical components necessarily to perform the various operations described herein. Devices such as smartphones, laptop computers, tablet computers, and wearable devices are generally collectively referred to as mobile devices.

The term “server” or “server device” is used to refer to any computing device capable of functioning as a server, such as a master exchange server, web server, mail server, document server, or any other type of server. A server may be a dedicated computing device or a server module (e.g., an application) hosted by a computing device that causes the computing device to operate as a server. A server module (e.g., server application) may be a full function server module, or a light or secondary server module (e.g., light or secondary server application) that is configured to provide synchronization services among the dynamic databases on computing devices. A light server or secondary server may be a slimmed-down version of server type functionality that can be implemented on a computing device, such as a smart phone, thereby enabling it to function as an Internet server (e.g., an enterprise e-mail server) only to the extent necessary to provide the functionality described herein.

As used herein, a “non-transitory machine-readable storage medium” or “memory” may be any electronic, magnetic, optical, or other physical storage apparatus to contain or store information such as executable instructions, data, and the like. For example, any machine-readable storage medium described herein may be any of random access memory (RAM), volatile memory, non-volatile memory, flash memory, a storage drive (e.g., hard drive), a solid state drive, any type of storage disc, and the like, or a combination thereof. The memory may store or include instructions executable by the processor.

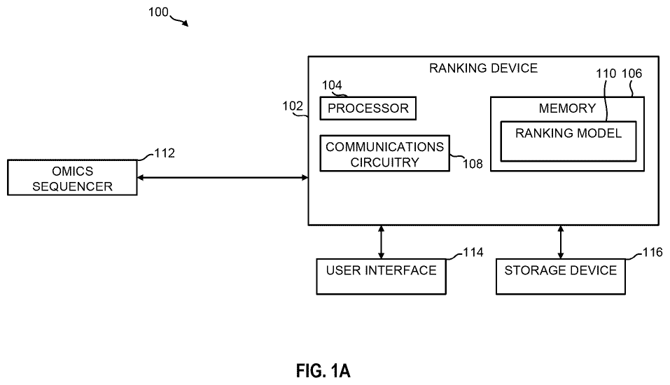

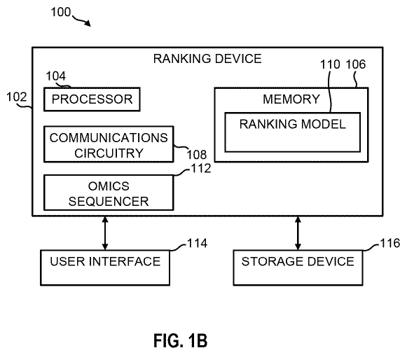

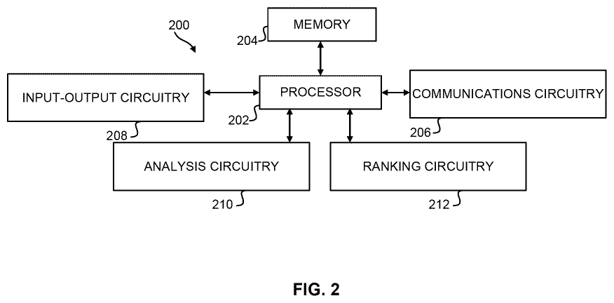

As used herein, a “processor” or “processing circuitry” may include, for example one processor or multiple processors included in a single device or distributed across multiple computing devices. The processor (such as, processor 104 and processing circuitry 202 shown in FIGS. 1A through 2) may be at least one of a central processing unit (CPU), a semiconductor-based microprocessor, a graphics processing unit (GPU), a field-programmable gate array (FPGA) to retrieve and execute instructions, a real time processor (RTP), other electronic circuitry suitable for the retrieval and execution instructions stored on a machine-readable storage medium, or a combination thereof.

As noted above, methods, apparatuses, systems, and computer program products are described herein that provide scores and/or ranking for analysis of a plurality of cells each applied with one of a plurality of active agents, each also at varying quantities, and/or with a mixture including a plurality of active agents. Traditionally, the output of OMICS sequencers or analyzers, such as a RNA sequencer or analyzer, may be difficult to interpret and may require user expertise or, at least, an experienced user. In addition, typically, such analysis and/or interpretation includes some level of bias, potentially skewing the end interpretation. Thus, the best active agent may not be selected by the end of such processes.

In contrast to these conventional techniques for determining which active agents to utilize in a formula or mixture, the present disclosure describes scoring and/or ranking a plurality of cells (for example, cells of interest) each applied with one of a plurality of active agents at a varying concentration. Such active agents may include cosmetic compounds. The cosmetic compounds may include one or more of peptides, molecules, extracts, chemicals, or devices. Further, the one or more of the plurality of active agents may include naturally occurring peptides. The devices may include one or more of a device that uses radiofrequency, fractional lasers, ablative lasers, non-ablative lasers, or micro needling. In an embodiment, the cells of interest may include one or more of skin cells, hair cells, scalp cells, tissue cells, reconstructed tissue, or ex vivo skin. Further, based on such scoring and/or ranking, the systems and methods described herein may determine an adjustment to a formula or suggest an amount of active agent to utilize in a particular formula. Such systems and methods may include or may connect to an OMICS sequencer or analyzer.

As the OMICS sequencer or analyzer analyzes a plurality of samples (in other words, a plurality of cells, each applied with an active agent and/or at varying concentrations), the resulting data may be transmitted to a computing device, scoring or ranking device or system, and/or a scoring or ranking apparatus. The OMICS sequencer may perform such an analysis in relation to or based on one or more characteristics of the cells, one or more gene characteristics, senescence scores, single cell transcriptomic measurements using RNA Multiplexed error-robust fluorescence in situ hybridization (MERFISH), and/or on another cell characteristic test, as will be understood by one skilled in the art. The OMICS sequencer may utilize one or more of genomics sequencing, transcriptomics sequencing, proteomics sequencing, epigenomics sequencing, lipidomics sequencing, or metabolomics sequencing or other types of OMICS sequencing or other tests to produce data. In other embodiments, the computing device, scoring or ranking device or system, and/or a scoring or ranking apparatus may obtain data from or via a user interface and/or from a database or other storage or memory. The computing device, scoring device or system, and/or a scoring apparatus may then score each of the analyzed cells. Based on those scores, the computing device, scoring or ranking device or system, and/or a scoring or ranking apparatus may determine an adjustment to a formula, provide a formula, and/or suggest a formula and/or adjustment to a formula. Further, the computing device, scoring or ranking device or system, and/or a scoring or ranking apparatus may, in addition to scores, rank the cells.

Accordingly, the present disclosure sets forth systems, methods, and apparatuses that provide easily interpretable analysis based on data from a OMICS sequencer, which is typically difficult to interpret. There are many advantages of these and other embodiments described herein. For instance, the resulting scores and/or ranking enable a user to easily determine the best active agent to utilize for a particular cellular characteristic. For example, a user may quickly and easily determine which peptide works best in relation to skin barrier health based on application of the peptide to skin cell samples and analysis thereof. In addition, the systems and methods described herein may provide suggested formula and/or adjustments to a formula based on the ranking and/or scoring.

Although a high level explanation of the operations of example embodiments has been provided above, specific details regarding the configuration of such example embodiments are provided below.

Example embodiments described herein may be implemented using any of a variety of computing devices or servers. To this end, FIG. 1A and FIG. 1B illustrate an example environment within which embodiments of the present disclosure may operate. As illustrated, a ranking system 100 may include a ranking device 102. The ranking device 102 may include a processor 104, a memory 106, and/or a communications circuitry 108. The memory 106 may include instructions and/or other software or algorithms, such as a ranking model 110. The ranking device 102 may connect, via the communication circuitry 108 and/or a communications network, to an OMICS sequencer 112, a user interface 114, and/or a storage device 116, among other devices and/or components. As illustrated and/or in other implementations, the ranking system 100, and any constituent device(s) and/or storage device(s) 116 may receive and/or transmit information via a communications network (for example, the internet and/or an intranet) with any number of other devices.

The ranking device 102 may be implemented as one or more servers, which may or may not be physically proximate to other components of the ranking system 102. Furthermore, some components of ranking device 102 may be physically proximate to the other components of the ranking system 100 while other components are not. Ranking device 102 may receive, process, generate, and transmit data, signals, and electronic information to facilitate the operations of the ranking system 100. Particular components of ranking device 102 are described in greater detail below with reference to apparatus 200 in connection with FIG. 2.

As noted, the memory 106 may include instructions and/or other algorithms. Such instructions, when executed by the processor 104, may control various functions and/or aspects of the ranking device 102. For example, a user may, via the user interface 114, initiate a scoring and/or ranking operation. Upon initiation and/or upon entry or input of one or more samples into the OMICS sequencer 112, the ranking device 102 may cause the OMICS sequencer 112 to analyze a plurality of cell samples. Each cell sample may be applied with a different active agent at a varying concentration and/or, in some embodiments, a mixture including a plurality of active agents with a selected concentration. The OMICS sequencer 112 may produce data corresponding to one or more characteristics, gene expressions (for example, enrichment, depletion, and/or other types of gene expressions), and/or other factors of the plurality of cells. For example, the OMICS sequencer 112 may produce a cell-by-gene matrix, gene expressions, and/or other data in various formats. For example, the OMICS sequencer 112 may generate skin barrier cell values upon analysis. In another embodiment, the ranking device 102 may receive or obtain data related to cell analysis from the storage device 116 and/or via the user interface 114. In another embodiment, the OMICS sequencer 112 may be included in or with or integrated into or with the ranking device 102, as illustrated in FIG. 1B. In an embodiment, the one or more characteristics may include one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health.

Upon reception of cell analysis data, the ranking device 102 may score and or rank each cell analysis via the ranking model 110. In an embodiment, the ranking model 110 may be based on a-score normalization, non-parametric or parametric statistical tests that are unpaired or paired, such as Wilcoxon rank sum tests and Wilcoxon signed-rank tests. In such examples, the Wilcoxon rank sum tests and Wilcoxon signed-rank tests may include non-parametric statistical tests that compare data that is not normally distributed (in other includes no variability within one or more groups. The ranking model 110 may utilize a hypothesized mean or median and a biological instance (for example, from the OMICS sequencer 112). The ranking device 102 may produce, generate, or determine a rank or score per each active agent at a selected concentration. Once scoring and/or ranking is complete, the ranking device 102 may determine a suggestion or adjustment to a formula including one or more of the active agents. Further, the suggestion or adjustment and/or the ranking or ranking may be displayed via the user interface 114.

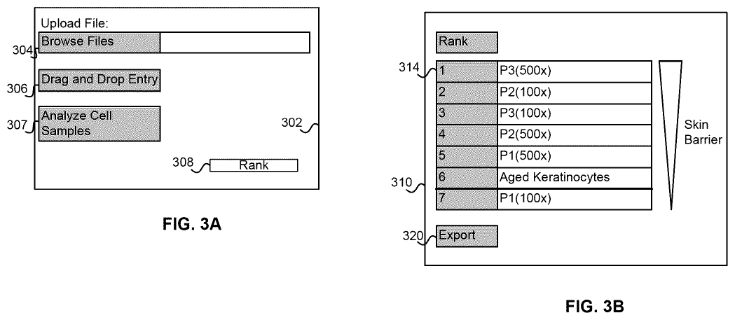

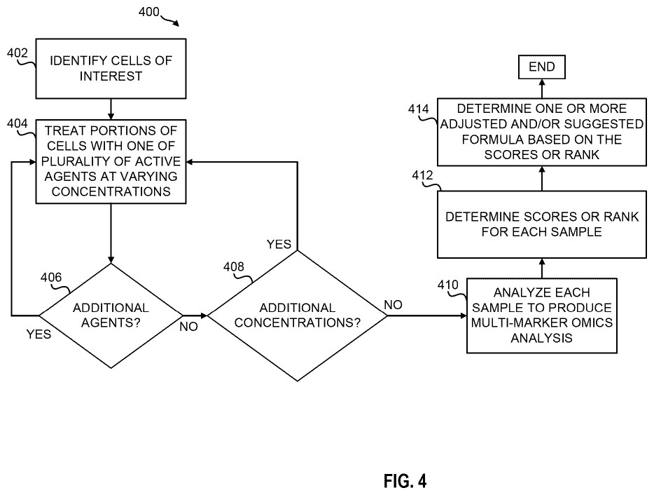

The ranking device 102 of the ranking system 100 (described previously with reference to FIGS. 1A and 1B) may be embodied by one or more computing devices or servers, shown as apparatus 200 in FIG. 2. As illustrated in FIG. 2, the apparatus 200 may include a processor 202, a memory 204, a communications circuitry 206, input-output circuitry 208, analysis circuitry 210, and ranking circuitry 212, each of which will be described in greater detail below. While the various components are only illustrated in FIG. 2 as being connected with processor 202, it will be understood that the apparatus 200 may further comprises a bus (not expressly shown in FIG. 2) for passing information amongst any combination of the various components of the apparatus 200. The apparatus 200 may be configured to execute various operations described above in connection with FIGS. 1A-1B and below in connection with FIGS. 3-4.

The processor 202 (and/or co-processor or any other processor assisting or otherwise associated with the processor) may be in communication with the memory 204 via a bus for passing information amongst components of the apparatus. The processor 202 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 200, remote or “cloud” processors, or any combination thereof.

The processor 202 may be configured to execute software instructions stored in the memory 204 or otherwise accessible to the processor (e.g., software instructions stored on a memory 106, as illustrated in FIGS. 1A-1B). In some cases, the processor may be configured to execute hard-coded functionality. As such, whether configured by hardware or software methods, or by a combination of hardware with software, the processor 202 represent an entity (such as, physically embodied in circuitry) capable of performing operations according to various embodiments of the present invention while configured accordingly. Alternatively, as another example, when the processor 202 is embodied as an executor of software instructions, the software instructions may specifically configure the processor 202 to perform the algorithms and/or operations described herein when the software instructions are executed.

Memory 204 is non-transitory and may include, for example, one or more volatile and/or non-volatile memories. In other words, for example, the memory 204 may be an electronic storage device (e.g., a computer readable storage medium). The memory 204 may be configured to store information, data, content, applications, software instructions, or the like, for enabling the apparatus to carry out various functions in accordance with example embodiments contemplated herein.

The communications circuitry 206 may be any means such as a device or circuitry embodied in either hardware or a combination of hardware and software that is configured to receive and/or transmit data from/to a network and/or any other device, circuitry, or module in communication with the apparatus 200. In this regard, the communications circuitry 206 may include, for example, a network interface for enabling communications with a wired or wireless communication network. For example, the communications circuitry 206 may include one or more network interface cards, antennas, buses, switches, routers, modems, and supporting hardware and/or software, or any other device suitable for enabling communications via a network. Furthermore, the communications circuitry 206 may include the processing circuitry for causing transmission of such signals to a network or for handling receipt of signals received from a network.

The apparatus 200 may include input-output circuitry 208 configured to provide output to a user and, in some embodiments, to receive an indication of user input. It will be noted that some embodiments will not include input-output circuitry 208, in which case user input may be received via a separate device such as a client device 112 (shown in FIG. 1). The input-output circuitry 208 may comprise a user interface, such as a display, and may further comprise the components that govern use of the user interface, such as a web browser, mobile application, dedicated client device, or the like. In some embodiments, the input-output circuitry 208 may include a keyboard, a mouse, a touch screen, touch areas, soft keys, a microphone, a speaker, and/or other input/output mechanisms. The input-output circuitry 208 may utilize the processor 202 to control one or more functions of one or more of these user interface elements through software instructions (e.g., application software and/or system software, such as firmware) stored on a memory (e.g., memory 204) accessible to the processor 202.

In addition, the apparatus 200 further comprises an analysis circuitry 210 that, in an embodiment, may initiate analysis of cell samples each applied with one or more active agents of varying concentrations. In another embodiment, the analysis circuitry 210 may include equipment or devices that perform the cellular analysis, such as transcriptomics and/or other tests to produce various gene expressions for each cell sample. In yet another embodiment, the analysis circuitry 210 may generate or determine a cell-by gene matrix based on data received from an analyzer or sequencer, such as an OMICS sequencer and/or other device or storage device. The analysis 210 may utilize processor 202, memory 204, or any other hardware component included in the apparatus 200 to perform these operations, as described in connection with FIGS. 1A-1B above and FIGS. 3-4 below. The analysis circuitry 210 may further utilize communications circuitry 206 to gather data from a variety of sources (such as, a storage device 116 or, as noted, an OMICS sequencer 112) and may utilize input-output circuitry 208 to receive data from a user (such as data related to cell analysis).

In addition, the apparatus 200 further comprises a ranking circuitry 212 that may score each of the active agents that have been applied to the analyzed cells and/or rank each of the active agents. The ranking circuitry 212 may utilize one or more statistical models to perform such a scoring and/or ranking. The ranking circuitry 212 may utilize a cell-by-gene matrix to generate the scores and/or ranking. In such an embodiment, the ranking circuitry 212 may first convert the cell-by-gene matrix into an array. The ranking circuitry 212 may then remove the first column in the array, the first column including, in an example, the agent identities. Next, the ranking circuitry 212 may generate an average of the values in the array and then define the number of instances in the array. The ranking circuitry 212 may then use a statistical model, such as the Wilcoxon sign-rank test, to generate the score and/or rank. The ranking circuitry 212 may utilize processor 202, memory 204, or any other hardware component included in the apparatus 200 to perform these operations, as described in connection with FIGS. 1A-1B above and FIGS. 3-4 below. The ranking circuitry 212 may further utilize communications circuitry 206 to gather data from a variety of sources (such as, from the analysis circuitry 210, storage device 116, and/or, as noted, an OMICS sequencer 112) and may utilize input-output circuitry 208 to receive data from a user (for example, the ranking circuitry 212 may receive data and/or a cell-by gene matrix from a user interface).

Although components 202-212 are described in part using functional language, it will be understood that the particular implementations necessarily include the use of particular hardware. It should also be understood that certain of these components 202-212 may include similar or common hardware. For example, the analysis circuitry 210 and ranking circuitry 212 may each at times leverage use of the processor 202, memory 204, communications circuitry 206, or input-output circuitry 208, such that duplicate hardware is not required to facilitate operation of these physical elements of the apparatus 200 (although dedicated hardware elements may be used for any of these components in some embodiments, such as those in which enhanced parallelism may be desired). Use of the terms “circuitry,” and “engine” with respect to elements of the apparatus therefore shall be interpreted as necessarily including the particular hardware configured to perform the functions associated with the particular element being described. Of course, while the terms “circuitry” and “engine” should be understood broadly to include hardware, in some embodiments, the terms “circuitry” and “engine” may in addition refer to software instructions that configure the hardware components of the apparatus 200 to perform the various functions described herein.

Although the analysis circuitry 210 and ranking circuitry 212 may leverage processor 202, memory 204, communications circuitry 206, or input-output circuitry 208 as described above, it will be understood that any of these elements of apparatus 200 may include one or more dedicated processor, specially configured field programmable gate array (FPGA), or application specific interface circuit (ASIC) to perform its corresponding functions, and may accordingly leverage processor 202 executing software stored in a memory (such as, memory 204), or memory 204, communications circuitry 206 or input-output circuitry 208 for enabling any functions not performed by special-purpose hardware elements. In all embodiments, however, it will be understood that the analysis circuitry 210 and ranking circuitry 212 are implemented via particular machinery designed for performing the functions described herein in connection with such elements of apparatus 200.

In some embodiments, various components of the apparatus 200 may be hosted remotely (such as, by one or more cloud servers) and thus need not physically reside on the corresponding apparatus 200. Thus, some or all of the functionality described herein may be provided by third party circuitry. For example, a given apparatus 200 may access one or more third party circuitries via any sort of networked connection that facilitates transmission of data and electronic information between the apparatus 200 and the third party circuitries. In turn, that apparatus 200 may be in remote communication with one or more of the other components describe above as comprising the apparatus 200.

As will be appreciated based on this disclosure, example embodiments contemplated herein may be implemented by an apparatus 200. Furthermore, some example embodiments may take the form of a computer program product comprising software instructions stored on at least one non-transitory computer-readable storage medium (for example, memory 204). Any suitable non-transitory computer-readable storage medium may be utilized in such embodiments, some examples of which are non-transitory hard disks, CD-ROMs, flash memory, optical storage devices, and magnetic storage devices. It should be appreciated, with respect to certain devices embodied by apparatus 200 as described in FIG. 2, that loading the software instructions onto a computing device or apparatus produces a special-purpose machine comprising the means for implementing various functions described herein.

Having described specific components of example apparatus 200, example embodiments of the present disclosure are described below in connection with a series of graphical user interfaces and flowcharts.

Turning to FIGS. 3A-3B, a graphical user interface (GUI) is provided that illustrates an interface enabling a user to input data, for example, a cell-by-gene matrix, and generate a ranking based on that data. As noted previously, a user may interact with the ranking system 100 by directly engaging with input-output circuitry 208 of an apparatus 200 comprising a ranking device 102 of the ranking system 100 and/or via the user interface 114. In such an embodiment, the GUIs shown in FIGS. 3A-3B may be displayed to a user by the apparatus 200 or the user interface 114.

Turning to FIG. 3A, a GUI 302 may include one or more inputs enabling a user to input data into the ranking system. For example, the GUI 302 may include a selectable button (see 304) that, when selected, generates a pop-up or separate window with a list of selectable files. In another embodiment, the GUI 302 may include a selectable button (see 306) that, when selected, generates a pop-up or separate window enabling a user to drag a file to the new pop-up or window for upload to the ranking system. In another embodiment, a user may initiate analysis of cell samples based on selection of button 307. In such an embodiment, the cell analysis may be performed once cell samples are received within an analyzer (for example, an OMICS sequencer). The GUI 302 may also include a selectable button (see 308) that when selected causes the ranking system to score and/or rank the received data.

Upon selection of button 308 and subsequent to ranking system scoring and/or ranking the data, a new GUI 310 may be generated. The GUI 310 may include a list of the active agents sorted according to rank (see 314). As way of illustration the GUI 310 includes data related to skin barrier health. The active agents in 314 may include a name of the active agent and the concentration of the active agent. In another embodiments, two or more active agents may be combined prior to application to a cell sample. The GUI 310 may include an export option (see 320). The export option may enable the user to download a copy of the ranking to a file location or desktop and/or to send the results to another user (for example, via e-mail).

Turning to FIG. 4, example flowcharts are illustrated that contain example operations implemented by example embodiments described herein. The operations illustrated in FIG. 4 may, for example, be performed by the ranking system 100 and/or the ranking device 102 of the ranking system 100 shown in FIGS. 1A-1B, which may in turn be embodied by an apparatus 200, which is shown and described in connection with FIG. 2. To perform the operations described below, the apparatus 200 may utilize one or more of processing circuitry 202, memory 204, communications circuitry 206, input-output circuitry 208, analysis circuitry 210, ranking circuitry 212, and/or any combination thereof. It will be understood that user interaction with the ranking system 100 may occur directly via input-output circuitry 208, or may instead be facilitated by a user interface 114, as shown in FIGS. 1A-1B, and which may have similar or equivalent physical componentry facilitating such user interaction. The order in which the operations are described is not intended to be construed as a limitation, and any number of the described blocks may be combined in any order and/or in parallel to implement the methods.

At block 402, a user or the ranking system 100 may identify cells of interest. The ranking system, for example, may receive a series of cell samples or a selected type of cells. In another embodiment, a user may input a series of cell samples to or into an analyzer. In another embodiment, identification of cells may include determining a type of cells for subsequent testing. For example, skin cells from a suitable subject may be selected when determining active agents for a product to protect skin. In a further embodiment, the skin cells may be selected from a selected part of the subject, for example, skin cells from the subject's face, around the subject's eyes, and/or from skin cell's exposed for prolonged period of time to an environment. In other embodiments, hair and/or scalp cells may be selected.

At block 404, once cells have been identified, each set of the cells may be treated with a concentration of one of a plurality of active agents and/or a mixture of a plurality of active agents. The user and/or ranking system may apply each of the active agents at a selected concentration to each distinct cell samples. Further, the active agents or the mixture may be applied iteratively at a selected concentration interval. The cell samples may then be input into the analyzer or sequencer, for example, an OMICS sequencer and/or other cell analysis or sequencer device.

Blocks 402 and 404 may be an iterative process. For example, a subject may provide numerous samples and active agents may be applied to each one of the cell samples in varying concentrations. At block 406, if additional active agents are available, then the those active agents may be applied to each of the cell samples. If, at block 408, additional concentrations of the active agents have not been utilized, then those active agents of the additional concentration may be applied to the remaining cell samples.

At block 410, once all the cell samples have been received or obtained, the cells may be analyzed by an analyzer or sequencer. Such an analysis may provide or generate a multi-marker OMICS analysis, a cell-by-OMICS matrix, and/or other data in one or more formats that include transcriptomics, proteomics, epigenomics, genomics, lipidomics, and/or metabolomics, among other characteristics. In an embodiment, prior to subsequent steps or processes, the data provided via such analysis may be further processed or converted to a format compatible to scoring or ranking by the ranking system. In other embodiments, the ranking system may convert data to a cell-by-gene matrix. In yet another embodiment, the ranking system may receive a cell-by-gene matrix from a user or other data source.

At block 412, the ranking system may determine scores for each cell sample and/or may rank each cell sample based on the applied active agents and/or mixture of active agents. The ranking system may utilize a statistical model and apply such data to the statistical model to generate the scores or ranking. The ranking system may utilize one or more statistical models to produce such scores or ranking.

Finally, at block 414, the ranking system may determine an adjustment to a formula and/or suggest a particular formula to achieve a desired result, such as, for example, an improved skin barrier health. The ranking system may determine such adjustments based on the ranking for each of a selected characteristic. For example, if a user desires to maximize skin barrier health and/or some other factor, then the ranking system may determine which of the active agents may provide a mixture that maximizes those aspects.

In another embodiment, the ranking system may generate a GUI to display the results of the ranking and/or the suggested formula. In such embodiments, the GUI may include buttons to export the results and/or the formula or adjustments to a formula. In another embodiment, the ranking system may include an option, via the GUI or via prompt, to include other cell samples applied with differing active agents in the current ranking or scoring. For example, a user may desire to include another sample within the current set of ranked or scored samples. Upon insertion of the sample into a OMICS Sequencer or upon reception of data corresponding to the new sample by the ranking system, the ranking system may re-rank or re-score the samples.

As described above, example embodiments provide methods and apparatuses that enable improved and easily interpretable analysis of cell sample. Example embodiments thus provide tools that overcome the problems faced by a user manually reviewing cell sample analysis, which requires significant time and expertise, while reducing potential human error (for example, miss-ranking or miss-scoring an active agent). Moreover, embodiments described herein avoid misinterpretation and eliminate the need for expertise in understanding selected cell analysis charts (such as a heat map). As these examples all illustrate, example embodiments contemplated herein provide technical solutions that solve real-world problems faced during interpretation of cell analysis.

FIG. 4 illustrates operations performed by apparatuses, methods, and computer program products according to various example embodiments. It will be understood that each flowchart block, and each combination of flowchart blocks, may be implemented by various means, embodied as hardware, firmware, circuitry, and/or other devices associated with execution of software including one or more software instructions. For example, one or more of the operations described above may be embodied by software instructions. In this regard, the software instructions which embody the procedures described above may be stored by a memory of an apparatus employing an embodiment of the present invention and executed by a processor of that apparatus. As will be appreciated, any such software instructions may be loaded onto a computing device or other programmable apparatus (e.g., hardware) to produce a machine, such that the resulting computing device or other programmable apparatus implements the functions specified in the flowchart blocks. These software instructions may also be stored in a computer-readable memory that may direct a computing device or other programmable apparatus to function in a particular manner, such that the software instructions stored in the computer-readable memory produce an article of manufacture, the execution of which implements the functions specified in the flowchart blocks. The software instructions may also be loaded onto a computing device or other programmable apparatus to cause a series of operations to be performed on the computing device or other programmable apparatus to produce a computer-implemented process such that the software instructions executed on the computing device or other programmable apparatus provide operations for implementing the functions specified in the flowchart blocks.

The flowchart blocks support combinations of means for performing the specified functions and combinations of operations for performing the specified functions. It will be understood that individual flowchart blocks, and/or combinations of flowchart blocks, can be implemented by special purpose hardware-based computing devices which perform the specified functions, or combinations of special purpose hardware and software instructions.

In some embodiments, some of the operations above may be modified or further amplified. Furthermore, in some embodiments, additional optional operations may be included. Modifications, amplifications, or additions to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A method for ranking a plurality of active agents applied to cells of interest, the method comprising:
identifying cells of interest of a subject;
iteratively treating different portions of cells with one of the plurality of active agents;
analyzing, via an OMICS analysis, each portion of treated cells to produce a multi-marker OMICS analysis;
determining a rank for each of the plurality of active agents for one or more selected characteristics based on application of the multi-marker OMICS analysis to an expression model for the one or more selected characteristics; and
determining one or more adjusted formula of a mixture including one or more of the plurality of active agents based on the rank of each of the plurality of active agents.

2. The method of claim 1, wherein determining the rank comprises:
converting the multi-marker OMICS analysis to an array of values, wherein the multi-marker OMICS analysis includes one of transcriptomics, proteomics, epigenomics, genomics, lipidomics, or metabolomics;
removing identifiers from the array of values;
determining an average of the array of values and a number of instances in the array of values;
applying the array of values to assigned pair or unpaired ranked test to generate a score for each value in the array of values based on a signature of the one or more selected characteristics; and
re-assigning the identifiers to the array of values.

3. The method of claim 1, wherein the plurality of active agents includes a plurality of cosmetic compounds; wherein the cosmetic compounds include one or more of peptides, molecules, extracts, chemicals, devices; wherein the devices include one or more of a device that uses radiofrequency, fractional lasers, ablative lasers, non-ablative lasers, or micro needling; and wherein one or more of the plurality of active agents comprise naturally occurring peptides.

4. The method of claim 1, further comprising:
in response to determination of the rank for each of the plurality of active agents:
generating a user interface;
displaying each of the plurality of active agents based on ranking; and
displaying each of the one or more adjusted formula.

5. The method of claim 1, wherein the cells of interest comprise one or more of skin cells, hair cells, scalp cells, scalp tissue, reconstructed tissue, or ex vivo skin.

6. The method of claim 1, wherein the one or more selected characteristics comprise one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health.

7. An apparatus for ranking a plurality of active agents applied to cells of interest, the apparatus comprising:
an analysis circuitry configured to:
obtain a multi-marker OMICS analysis of a plurality of portions of treated cells, each of the portions of treated cells being treated with one or more of a plurality of active agents; and
a ranking circuitry configured to:
determine a rank for each of the plurality of active agents for one or more selected characteristics based on application of the multi-marker OMICS analysis to an expression model for the one or more selected characteristics, and
determine one or more adjusted formula of a mixture including one or more of the plurality of active agents based on the rank of each of the plurality of active agents.

8. The apparatus of claim 7, further comprising an OMICS sequencer configured to analyze cells to produce the multi-marker OMICS analysis, and wherein the OMICS sequencer includes an RNA sequencer and the multi-marker OMICS analysis includes multi-marker genetic analysis.

9. The apparatus of claim 8, wherein the analysis circuitry communicatively connects to the OMICS sequencer and wherein the analysis circuitry is further configured to, in response to reception of cells of interest at the OMICS sequencer, prompt the OMICS sequencer to analyze the cells of interest.

10. The apparatus of claim 7, further comprising a user interface communicatively connected to the analysis circuitry and wherein the analysis circuitry obtains the multi-marker OMICS analysis from the user interface.

11. The apparatus of claim 7, wherein the active agents include naturally derived peptides, wherein the cells of interest comprise skin cells, and wherein the one or more selected characteristics comprise skin barrier health.

12. A computer program product for ranking a plurality of active agents applied to cells of interest, the computer program product comprising a non-transitory machine-readable storage medium storing software instructions that, when executed, cause an apparatus to:
obtain a multi-marker OMICS analysis of a plurality of portions of treated cells, each of the portions of treated cells being treated with one or more of a plurality of active agents; and
determine a rank for each of the plurality of active agents for one or more selected characteristics based on application of the multi-marker OMICS analysis to an expression model for the one or more selected characteristics.

13. The computer program product of claim 12, wherein the non-transitory machine-readable storage medium stores additional software instructions that, when executed, cause an apparatus to determine one or more adjusted formula of a mixture including one or more of the plurality of active agents based on the rank.

14. The computer program product of claim 13, wherein the mixture comprises a topical mixture applicable to skin and including one or more of the plurality of active agents and other materials.

15. The computer program product of claim 13, wherein multi-marker OMICS analysis comprises one or more of a heatmap, a cell by gene matrix, or cell type distribution.
